(19) United States
(12) Patent Application Publication
(10) Pub. No.: US 2026/0083494 A1
(43) Pub. Date: Mar. 26, 2026

(54) MULTI-CONTACT ELECTRODE AND INTEGRATED CIRCUIT FOR S-EEG

(71) Applicant: Adept Neuro SA, Lausanne (CH)

(72) Inventors: Komail Badami, Oberengstringen (CH); Amrith Sukumaran, Buchs (CH); Stéphane Emery, Stallikon (CH); Jinyu Jason Ruan, Pully (CH)

(73) Assignee: Adept Neuro SA, Lausanne (CH)

(21) Appl. No.: 19/109,349

(22) PCT Filed: Sep. 6, 2022
(86) PCT No.: PCT/IB2022/058366

§ 371 Date: Mar. 6, 2025

(51) Int. Cl. A61B 18/14; A61B 18/00

(52) U.S. Cl. CPC A61B 18/1477; A61B 2018/00446; A61B 2018/00589; A61B 2018/00791; A61B 2018/00839; A61B 2018/1467

(57) ABSTRACT

A multi-contact electrode device, comprising an elongated body with plurality of pick-up contacts and a slender ASIC into a hollow lumen. The ASIC is configured to readout, process and digitize the EEG signals present at the contacts and to apply selectively a high RF current to any selected pair of contacts for thermocoagulation. The ASIC also includes a microcontroller with a digital communication interface.

### TECHNICAL DOMAIN

The present invention concerns a multi-contact electrode for medical use. Embodiments of the invention relate to intracerebral electrodes that are intended to be placed in the brain of a patient for diagnostic and therapeutical procedures such as stereotactic electroencephalography (S-EEG), but the invention can be applied to other instrumental medical procedures as well.

### RELATED ART

Multi-contact electrodes are used for electrophysiological exploration of several structures in the body, especially in the central nervous system. Stereotactic electroencephalography (S-EEG), for example relies on electrodes penetrating in the brain that measure the electrophysiological activity of deep structures that are not easily accessible by superficial measurement techniques such as transcranial electroencephalography (EEG) and electrocorticography (ECOG). Stereotactic electroencephalography is considered less invasive than electrocorticography, as it only requires small-diameter burr holes in the cranium, rather than a more extensive craniotomy.

Existing S-EEG readout employ thin passive S-EEG electrodes (diameter of about 1.5 mm) connected to a processing and visualizing station that is typically at a few metres distance through thin wires. The number of conductors is directly proportional to the number of recording sites inside the patient's brain, otherwise said, to the number of electric contacts, and may be in a range of 4-20 wires per electrode.

Document US 2012/0296404 A1 discloses a multi-electrode intracerebral electrode for placement in a patient's brain. The electrode has a plurality of contact pads that take the form of metallic rings on the side of an elongated catheter.

Besides recording the electric activity of live tissues, elongated electrodes are also used in thermocoagulation procedures, where the electrodes are used to inject a radiofrequency current that destroys or inactivates a targeted region of tissue. These procedures have been proposed and used in the treatment of drug-resistant seizures, as disclosed in IN 2002011001043, in controlling pain of spinal origin, and in the treatment of other conditions.

These known devices present a common shortcoming: the weak electric signals collected by the electrode are transmitted by thin conductors to an external electronic processor. The wires may pick up unwanted signals, for example the mains signal at 50/60 Hz and signals induced by the unavoidable motion of the patient and of the cables, that interferes with the interpretation of the true electrophysiological signal. Thin wires, moreover, are ill suited to carry the RF at the current level needed in thermocoagulation procedures, and there is a limit to the number of conductors that can be find place in the lumen of a catheter of a reasonable gauge.

U.S. Pat. No. 9,381,063 B2 discloses a magnetic-guided soft catheter for renal nerve ablation. This device has at its proximal end a transistor circuit for processing electrophysiological signals.

### SHORT DISCLOSURE OF THE INVENTION

An aim of the present invention is the provision of a device that overcomes the shortcomings and limitations of the state of the art.

According to the invention, these aims are attained by the object of the attached claims, in particular by a multi-contact electrode device, comprising an elongated body with a distal end for placement into a target region of a patient's body, the device comprising a plurality of pick-up contacts on an active portion of the elongated body adjacent the distal end for recording an electrical activity of the target region and for delivering a radiofrequency current into the target region, the elongated body containing, in the active portion, an integrated circuit that comprises: a readout unit with a plurality of input channels, configured for amplifying and digitizing electrical signal impressed on at least some of the pick-up contacts by the electrical activity of the target region, a thermocoagulation unit receiving a radiofrequency signal, selectively connectable to at least one selected contact out of the pick-up contacts for injecting a localized radiofrequency current into the target region, a digital communication interface, and by an especially configured integrated circuit as detailed above with two linear dimensions lower than 2 mm, preferably lower than 1 mm, more preferably equal to or lower than 0.75 mm.

Dependent claims relate to important and advantageous optional features, such as the fact that the thermocoagulation unit is configured to handle a current of at least 50 mA rms, preferably more than 120 mA rms and a voltage of at least 15 V rms, preferably more than 20 V rms at the selected contact, the presence of a readout channel for a temperature sensor, pick-up contacts made of conductive areas on a flexible printed circuit that is wrapped around the active portion, placing the integrated circuit is in a hollow lumen of the elongated body, possibly through a longitudinal slit in the same, ten or more readout channels, preferably differential, high-pass filters with a cut-off frequency lower than 0.2 Hz.

Other optional features of the invention include: a wireless digital communication interface, generation of the electrocoagulation RF signal in the electrode, by a DC/AC converter, or generation of the voltage supply needed by the integrated circuit in the electrode, by an AC/DC converter, an automatic classification of the S-EEG signals based on an algorithm implemented in the integrated circuit. These features are intended to reduce the number of wires to each electrode and to simplify and reduce the complexity of external devices.

With respect to what is known in the art, the invention provides several advantages. On the one hand, the weak electric signals picked up by the contacts are amplified processed and converted in a robust digital format in the distal end of the electrode, rather than carried by thin wires to an external processing assembly. Further, the number of wires is no longer proportional to the number of readout sites, since the data can travel on a digital bus in a serial format. This reduces the number of cables running to the electrode, eliminates most of the electronic pick-up noise and of the power losses that occur in the wires during the thermocoagulation procedure.

The electronic processing of the data is simplified since the data are pre-processed in the electrode itself.

The electrode of the invention is suitable for being employed in various medical procedures in the central nervous system, in the spine, or in other districts. The present disclosure will refer only, for brevity's sake, to a S-EEG procedure, being it intended that this is not a limitation of the invention.

### SHORT DESCRIPTION OF THE DRAWINGS

Exemplar embodiments of the invention are disclosed in the description and illustrated by the drawings in which:

FIG. 1 illustrates schematically the active region of the electrode.

FIGS. 2 and 3 show an application-specific integrated circuit (ASIC).

FIG. 4 show schematically a possible structure of the application-specific integrated circuit.

FIG. 5 illustrates, in conceptual fashion, an apparatus for S-EEG investigation and thermocoagulation using the inventive electrodes.

FIGS. 6 and 7 show a section of the active region of the electrode in two examples of realization.

### EXAMPLES OF EMBODIMENTS OF THE PRESENT INVENTION

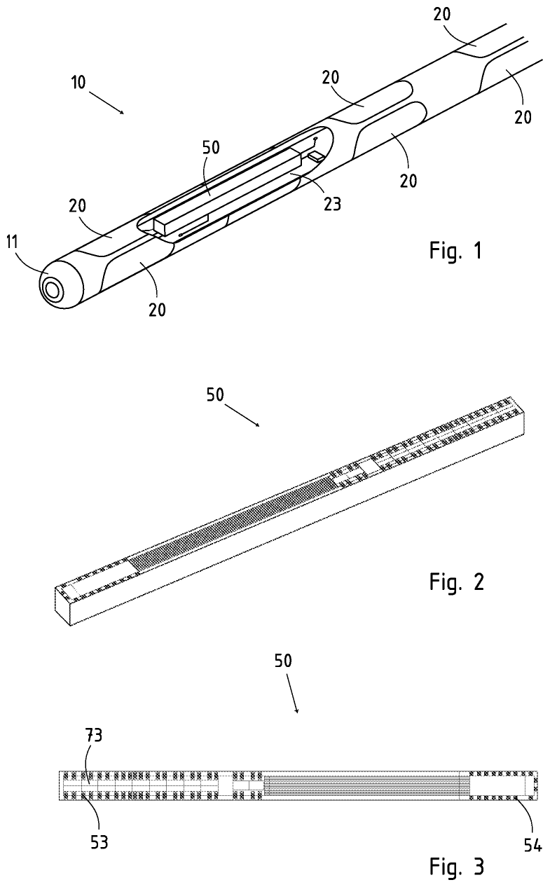

FIG. 1 shows the active, distal part of a S-EEG deep electrode according to the invention. The electrode has a tip 11, corresponding to the distal end, and an elongated body 10. A cylindrical shape is customary, but other sections, for example flattened or prismatic, are not excluded.

Several electrical contacts 20 are disposed along a side surface of the elongated body 10. Their number is not an essential limitation of the invention. In a possible example, the electrode has eighteen contacts, but the number may be lower or higher, according to the needs. The diameter of the body 10 may be of 3 mm or less.

The elongated body 10 is drawn partially transparent to show that it contains a flexible printed circuit board 23 with an application-specific integrated circuit 50, that will be further described later. Importantly, the circuit is fabricated on a semiconductor die that has at least two linear dimensions-width and thickness, for example-less than the transverse dimension of the electrode. In a possible implementation, the electrode is hollow and has an inner lumen that contains the integrated circuit. In other realizations, the lumen could be filled with a suitable material after the insertion of the integrated circuit. In examples, the thickness and width of the die are less than 2 mm, preferably less than 1 mm, more preferably equal to or less than 0.75 mm. In an embodiment, the dimensions of the die are 0.75 mm×0.75 mm×14 mm, yielding an aspect ratio of approximately 1÷18.

The limited space available in the lumen requires an especially compact package for the integrated circuit 50, preferably a no-lead package. To further save space, the integrated circuit 50 may be a bare die, for example a flip-chip circuit, directly soldered on a flexible printed circuit 23.

FIGS. 2 and 3 show a possible realization of the integrated circuit 50 in a flip-chip package. The circuit has a very slender form factor and a plurality of contact pads 53, 54 on one surface, connectable to the printed circuit by solder bumps. Contact pads 53 are dimensioned to carry higher currents and can withstand higher potential. They are used in radiofrequency thermocoagulation procedures. Close to the thermocoagulation contacts 53 is an array of high-current switches 73 that are used to select a pair of contacts in a thermocoagulation procedure.

The aspect ratio (length over width) of the chip of the integrated circuit 50 is preferably higher than 10, more preferably higher than 15. The chip in the example has a width of 750 um and a length of 14 mm. The thickness is 750 μm, which is a standard value proposed by many semiconductor foundries and is rather easy to manipulate. Accidentally, this thickness is the same as the width of the chip in this example. The thickness could be reduced, for example to 300 μm or 100 μm, although this would require more careful manipulation and possibly the use of carriers. The chip could also be thinned by an abrasive step, before or after its soldering.

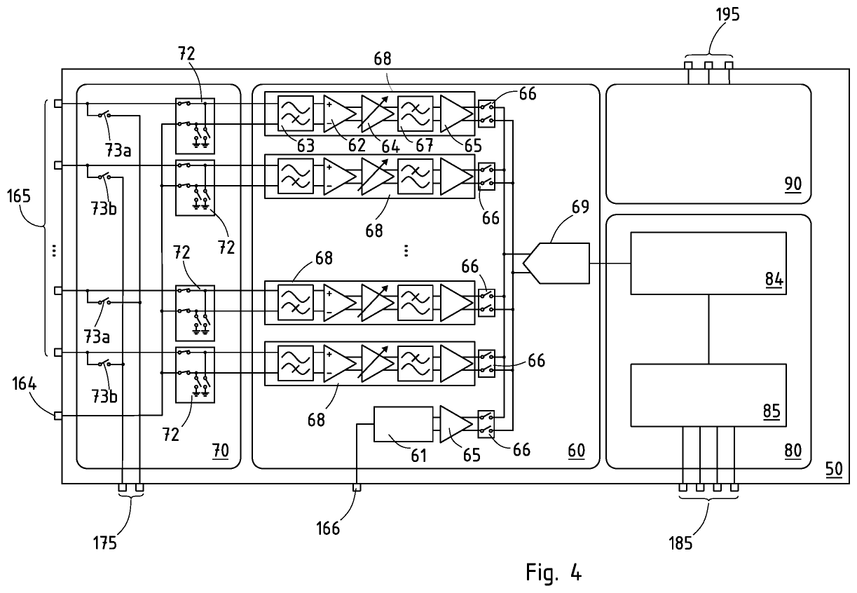

FIG. 4 is a conceptual representation of the subunits composing the integrated circuit 50, in an embodiment. The readout unit 60 receives the weak bioelectric signals present at the contacts 20, which are electrically connected to the input terminals 165 of the IC and convert them into a suitable digital representation. As mentioned, the number of contacts is not determined in the invention but, for example, the electrode of the invention may have eighteen pick-up contacts 20, organized in 9 pairs along the electrode, and the integrated circuit 50 may have eighteen input terminals 165 for reading 18 signals from the same contacts 20. FIG. 4 shows only four readout channels for simplicity.

The readout unit comprises an analog-to-digital converter 69 that reads a plurality of input channels in a time-multiplexed fashion determined by the sequential commutation of the switches 66. The sampling frequency will be chosen according to the bandwidth of the signals of interest and may be programmable.

The ADC 69 may be implemented as a successive approximation converter, a monotonic-switching successive approximation converter, a sigma-delta ADC, or in any other suitable manner. Much important information of EEG signal is contained in the lower part of the frequency spectrum, at or below 10 Hz, but higher-frequency components also have clinical importance, and it is advantageous to oversample the signals to extract their fine features and filter out the noise. In a practical embodiment, the ADC may run at 2 Ms/s and the sampling rate on each channel will be 100 kHz, assuming a multiplexing factor of 20÷1.

An analogue processing front end 68 is present between each input terminal 65 and the corresponding port in the multiplexer 68. FIG. 4 shows only four front end channels for simplicity, but the integrated circuit 50 may include several more, possibly identical, as needed to acquire the signals of all the contacts 20.

In the presented example, the analogue front ends are differential and are all referred to a common reference terminal 164. The multiplexing switches 66 are also differential, as the analog input of the converter 69. This differential topology improves rejection of common mode noise but is not an essential feature of the invention.

Each processing channel includes a low-noise amplifier 62 that is followed in this example by a programmable-gain amplifier 64 whose amplification factor can be set by the control unit 80, a low-pass filter 67 and a buffer 65 before the multiplexing switch 66, The low-pass filter 67 may be for example an anti-aliasing low-pass filter, or a more complex filter. In an embodiment, the bandwidth of the analogue processing channel may be of 5 kHz, and the corner frequency of the low-pass filter may be programmable by the control unit 80, according to the needs, to 5 kHz, 1 kHz, or other desired values. Although the low pass filter 67 is represented by a separate block in the drawing, the desired transfer function may be provided intrinsically by the programmable amplifier 64 or another element.

The overall gain of each analog front end is preferably programmable by the control unit 80 and may reach 57 dB or more. Preferably, the gains of the individual front ends are equalised to a common nominal value through trimmable elements in the integrated circuit 50.

Assuming a sampling rate of 100 kHz, the EEG signals are heavily oversampled, which helps extracting their fine features. Buffers 65 are useful to drive the input of the ADC 69 and the parasitic impedance of the inactive channels, which appear as a capacitive load of a few pF.

In clinical reality, the EEG signal is generally superposed to a slowly drifting DC voltage. Analog channels 68 preferably also include a high pass filter 63 to suppress this DC component. This high pass filter should have a very low cut-off frequency to preserve the slow components of the EEG signal, for example a cut-off frequency lower than 0.2 Hz, or between 0.1 Hz and 0.2 Hz. The high pass filter can be realised in any suitable way, for example as passive C-R filter, where the resistive element is obtained in the integrated circuit with a MOS pseudo resistor. The low-noise amplifier 62 should have a high input impedance to avoid loading the high pass filter. Additional interstage high-pass filters (not drawn) may be used to remove the DC offset from the amplifiers.

In another realization, not represented in the drawings, the analogue front ends 68 could be DC coupled, and the DC offset mitigated or compensated by subtracting a suitable correction signal, in a feedback configuration. The feedback loop could include a mixed analogue/digital circuit or a switched capacitor circuit. This approach may provide a transfer function like that of a very low frequency high pass filter avoiding the large decoupling capacitors needed by passive filters and the nonlinearities of some pseudo resistors.

The readout unit 60 may have additional input channels that acquire other analogue signals at the same time as the EEG signals present at the terminals 165. These additional channels will be designed to cope with the dynamics of the respective signals. For example, the integrated circuit 50 may have a terminal 66 for an electric thermometer, such as a thermistor, a bandgap semiconductor temperature sensor, or any other suitable transducer to read body temperature. A special processing circuit 61 is configured to provide an analogue temperature signal compatible with the A/D converter 69.

Thermocoagulation is achieved by applying a radiofrequency high-voltage signal across two intended electrodes. The thermocoagulation unit 70 receives a high-voltage radiofrequency signal from a suitable source, which can be an approved medical device for the RF thermocoagulation and is connected to terminals 175. Typically, the signal present at the terminals 175 may be a sinusoid with a frequency of 480 kHz and a differential amplitude of 64 V pp, or about 22 V rms. The thermocoagulation unit 70 is configured as a switching matrix that can connect selectively the high-voltage source to one or more selected terminals 65 through the switches 73a and 73b.

Importantly, the analogue front ends 68 are connected to the corresponding input terminal through isolation switches 72 that can be operated by the on-board controller 80 during a thermocoagulation procedure. In this way, the analogue front ends 68 are protected from the high voltages used during the thermocoagulation. To increase the attenuation, the isolation switches 72 are configured to open the connection between the input of the analogue channels and the terminals 165 and, at the same time, short the inputs of the analogue channels to ground.

The switches 73a and 73b are open during the EEG readout and can be selectively closed by the controller 80 to apply the RF voltage to a pair of selected contacts. Possibly, a pair of successive switches is closed, to apply the RF voltage to a pair of adjacent contacts, but other schemas are possible. The switches should present a low resistance in the “on” state since the currents involved are considerable. The inventors have determined that a resistance of about or less than 20Ω is a favourable compromise. This value is well below the combined resistance of the supply conductors and of the nervous tissue that is treated. Even lower resistance would lead to lower losses but would take more silicon area. The radiofrequency current injected in the tissue will be at least 50 mA, preferably more than 120 mA rms and the voltage drop between the selected contacts will be at least 15 V rms, for example 22 V rms. The switches included in the thermocoagulation unit 70 are configured and dimensioned to handle such levels of voltage and current.

The control signal needed to operate the isolation switches 72 and the selection switches 73a, 73b are generated by the logic unit 80 sfollowing a predefined program and/or commands received through the data bus interface 85. The logic circuit 80 operates at a much lower voltage level than the high-voltage selection matrix 70, and the integrated circuit 50 may include level shifters (not represented in the drawings) for raising the control signals from the voltage domain of the logic circuit to that of the thermocoagulation unit. Typically, the logic levels of control signals may be 0 and 1.8V, which is shifted to ±16V or more.

In another, non-represented, variant, the high-voltage radiofrequency signal could be generated by a suitable RF generator in the multi-contact electrode, or possibly on the same integrated circuit 50, rather than by an external unit.

The integrated circuit 50 also includes a power management unit 90, that receives electric power from terminals 195 and provides DC voltages needed to power the other elements of the integrated circuit.

Possibly, the power management unit will receive an external DC supply, for example +3.3 VDC, from which stable voltages and currents required by the logic unit 80 and the readout unit 60 are derived. The power management unit may include a bandgap reference, or another stable voltage reference to ensure that the generated voltages correspond to the desired levels. The power supply is bypassed by external capacitors connected to dedicated terminals, not shown in the drawings.

In a variant, the power management unit 90 could include an AC/DC converter uses the RF AC signal present at the terminals 175 to provides the needed DC voltages. In this case, the number of wires per electrode is reduced.

The control and communication unit 80 that has a logic circuit 84, through which all the functions of the integrated circuit 50 can be controlled remotely, and an interface 85 for a communication bus 185, for example an SPI bus. The logic circuit 84 may be a finite state machine, a microcontroller, or any other suitable logic circuit. The control and communication unit may also include a digital memory to store the values generated by the ADC 69 before their transmission through the data bus 185.

Even if the functions of the integrated circuit 50 have been described using separate blocks, this is a conceptual analogy that may, or may not, be followed in a concrete hardware implementation of the invention. The integrated circuit 50 may include separate circuit areas that correspond to the readout unit 60, the radiofrequency unit 70, the power management unit 90, the control and communication unit 80. Yet, in other embodiments of the invention, the circuit 50 may be organized differently.

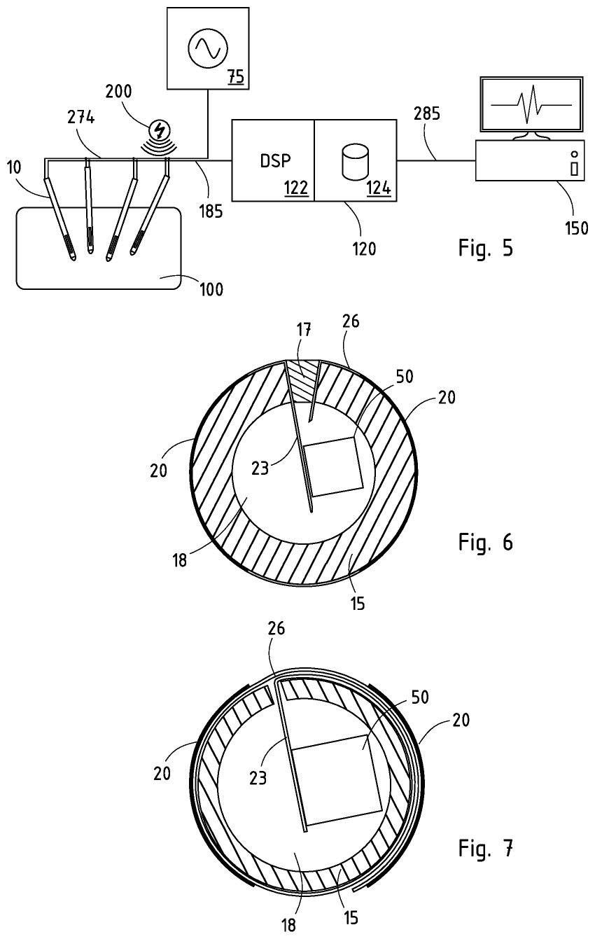

FIG. 5 shows a possible setup for a S-EEG procedure where a certain number of electrodes 10 is implanted in a target area 100, for example a chosen structure in the brain of a patient. The electrodes are connected to an interface unit 120 via the digital bus 185, and the EEG signals picked up by the contacts 20 are transmitted to the interface 120 as robust digital signals, immune to the electromagnetic disturbances 200 (for example induced signals from the mains network at 50/60 Hz). Moreover, even if the number of active channels may be large, the data are transmitted on a limited number of conductors. It is possible then to adopt thinner electrodes and/or increase the gauge of the conductors.

FIG. 5 shows the RF source 75 used for the thermocoagulation procedures, connected to the electrodes 10 with a separate line 274. The electrodes may receive also a low-voltage power supply via another line that is not represented.

In a possible variant of the invention, the wired bus 85 may be replaced by a wireless data communication channel, for example Bluetooth®, and the number of wires could be further reduced. In an especially advantageous variant, the communication is wireless, and the power management unit is supplied by the RF signal itself, such that the only wired connection required would be the line 274 carrying the RF signal. Should, instead, the chip 50 be capable of generating the high-voltage RF signal needed for the thermocoagulation, the external generator 75 and the corresponding line 274 would no longer be needed.

The integrated circuit may include additional functions including an automatic classification of the S-EEG signals based on an algorithm, preferably implemented in the integrated circuit 50. This may include a machine-learning system, a neural network, or any suitable automatic classification program. Possibly, the multi-contact electrodes 10 may carry out a pre-classification which could be improved and finalised in the external processor 120.

The interface 120 is considerably simplified when compared to what is needed in conventional S-EEG procedures, because a good deal of processing is carried out in the integrated circuits 50 in the electrodes 10. The interface 120 may perform a digital signal processing of the signal, for example with advanced digital filters to make the desired signals stand out of the noise or classify the EEG signals. In its simplest realization, however, the processing is entirely carried out in the ASICS 50 of the electrodes 10 and the interface 120 acts mostly as storage and concentrator. An analysis workstation 150, which may be a personal computer running a specialised program, interacts with the interface via a suitable network link 185, for example an IP link.

FIG. 6 shows a section of the inventive electrode in the active region with a tubular body encircling a hollow lumen 18. A flexible circuit 23 enters the lumen 18 through the slit 17 and is wrapped around the tubular mandrel 15. The pick-up contacts 20 on the surface of the circuit 23 are exposed on the side of the electrode, while the integrated circuit 50 is in the interior lumen. The flexible circuit 23 may be realised out of a polyimide film or any other suitable material. The flexible circuit may be bonded adhesively to the surface of the mandrel 15. If required, the slit 17 can be closed once the flexible circuit 23 is in place, to seal the inner lumen 18.

FIG. 7 shows an alternative realization in which the flexible circuit 23 is rolled on itself by more than one turn providing an uninterrupted cylindrical surface 26 without apertures. A hollow mandrel 15 can be used, but it might be omitted if the rolled flexible circuit provides a sufficient stiffness.

### REFERENCE SYMBOLS IN THE FIGURES

10 elongated body
11 distal end, tip
15 tubular body
17 slit
18 lumen
20 pick-up contact
23 flexible printed circuit
26 side surface
50 application-specific integrated circuit
53 contact pad, high voltage and current
54 contact pad, low voltage and current
60 readout unit
61 temperature front-end
62 low-noise amplifier
63 high-pass filter
64 programmable-gain amplifier
65 buffer, AB class driver
66 multiplexing switch
67 low-pass filter
68 readout channel
69 A/D converter
70 thermocoagulation unit
72 isolation switch
73a/b selection switch, odd/even channels
75 high-voltage radiofrequency generator
80 logic and communication unit
84 logic unit, microcontroller, finite state machine, data buffer
85 digital communication interface
90 power management unit
100 target region
120 interface
122 digital signal processing
124 storage
150 analysis station
165 input channels
166 temperature transducer terminal
175 high-voltage input terminals
185 data bus terminals
274 high-voltage cable
285 network

1. A multi-contact electrode device, comprising an elongated body with a distal end for placement into a target region of a patient's body, the device comprising a plurality of pick-up contacts on an active portion of the elongated body adjacent the distal end for recording an electrical activity of the target region and for delivering a radiofrequency current into the target region, the elongated body containing, in the active portion, an integrated circuit that comprises:
a readout unit with a plurality of input channels, configured
for amplifying and digitizing electrical signal impressed on at least some of the pick-up contacts by the electrical activity of the target region, p1 a thermocoagulation unit receiving a radiofrequency signal, selectively connectable to at least one selected contact out of the pick-up contacts for injecting a localized radiofrequency current into the target region,
a digital communication interface.

2. The device of claim 2, wherein the integrated circuit has two 20 more preferably equal to or lower than 0.75 mm.

3. The device of claim 1, wherein the thermocoagulation unit is configured to handle a current of at least 50 mA rms, preferably more than 120 mA rms and a voltage of at least 15 V rms, preferably more than 20 V rms at the selected contact.

4. The device of claim 1, including a readout channel for a temperature sensor.

5. The device of claim 1, wherein the pick-up contacts are conductive areas on a flexible printed circuit that is wrapped around the active portion.

6. The device of claim 1, wherein the integrated circuit is in a hollow lumen of the elongated body.

7. The device of claims 5, the elongated body having, in the active portion, a longitudinal slit allowing a part of the printed circuit to enter the lumen.

8. An integrated circuit comprising,
a readout unit with a plurality of terminals connectable to pick-up contacts, configured for processing and digitizing bio-electrical signals picked up by the pick-up contacts,
an input for a radiofrequency signal, selectively connectable to at least one selected terminal from the terminals for injecting a localized radiofrequency current into a target region near a pick-up contact,
a digital communication interface,
wherein the integrated circuit has two linear dimensions lower than 2 mm, preferably lower than 1 mm, more preferably equal to or lower than 0.75 mm.

9. The integrated circuit of claim 8, configured to handle a radiofrequency signal having a current of at least 50 mA rms, preferably more than 120 mA rms and voltage of at least 15 V rms, preferably more than 20 V rms at the selected pick-up contact.

10. The integrated circuit of claim 8, including a readout channel for a temperature sensor.

11. The integrated circuit of claim 8, the readout unit including ten or more readout channels configured to process a plurality of bioelectric signals from distinct pick-up contacts.

12. The integrated circuit of claim 8, the readout unit including differential readout channels configured to process bioelectric voltage signals relative to a reference potential.

13. The integrated circuit of claim 8, the readout unit
including one or more high-pass filters with a cut-off frequency lower than 0.2 Hz.
